(19) United States
(12) Patent Application Publication
(10) Pub. No.: US 2026/0114773 A1
(43) Pub. Date: Apr. 30, 2026

(54) MULTI-USE DYNAMOMETER

(71) Applicant: VALD PTY LTD, Queensland (AU)

(72) Inventors: Sam JAMES, Queensland (AU); Jason SMITH, Queensland (AU)

(21) Appl. No.: 19/118,563

(22) PCT Filed: Oct. 5, 2022
(86) PCT No.: PCT/AU2022/051191

§ 371 Date: Apr. 4, 2025

(51) Int. Cl. A61B 5/22; A61B 5/00; A61B 90/00; A63B 24/00; G01C 21/16

(52) U.S. Cl. CPC A61B 5/225; A61B 5/6825; A63B 24/0062; G01C 21/16; A61B 2090/064; A61B 2560/0431; A61B 2560/045; A61B 2562/0219

(57) ABSTRACT

A modular, multi-use dynamometer 10 having a body 100 with a connector 120, 140 at each end for releasable attachment of accessories, such as a pads, loops or hooks, or a grip strength tool. The dynamometer can measure both compression and tensile forces. The accessories may have an electronically readable element, such as NFC or RFID such that the dynamometer 10 knowns which accessories are attached and reacts accordingly. The dynamometer preferably also has an in built IMU to allow range of motion measurements and the like.

### FIELD OF THE INVENTION

The invention relates to a dynamometer. In particular, the invention relates, but is not limited, to a multi-use dynamometer that can be hand held to measure both tension and compression.

### BACKGROUND TO THE INVENTION

Reference to background art herein is not to be construed as an admission that such art constitutes common general knowledge.

In various settings, such as medical, physiological, rehabilitation, kinesiology, it is helpful to be able to measure the strength of a patient or subject. Different types of dynamometers are often used in medical, physical therapy, and/or physiotherapy settings to measure muscle strength. However, a variety of dynamometers are required to take a comprehensive set of measurements of a subject or, even just to be able to provide the option to take a wide variety of measurements.

For example, one type of dynamometer, often called a grip dynamometer, measures the amount of force a user can produce with their grip, e.g. by pulling with their fingers. Such dynamometers typically have a frame and measure tension forces between the body of the dynamometer, supported by the palm of the user via the frame, and a grip which the user pulls towards the portion of the frame engaging with their palm.

Another type of dynamometer measures muscle strength by measuring the amount of force a user can apply to a pad, or the like. Such dynamometers measure compression between the body of the dynamometer and the pad. Different dynamometers have different sized and shaped pads. Some also have replaceable pads to allow fitment of different sized and shaped members using the same dynamometer.

Although having different shaped pads or members with a single dynamometer allows a single dynamometer to take a wider variety of measurements, it is up to the practitioner to ensure the measurements are taken and recorded correctly. A relatively easy mistake is to take measurements using different pads but record one or more of the measurements against an incorrect exercise or muscle test.

### OBJECT OF THE INVENTION

It is an aim of this invention to provide a dynamometer which overcomes or ameliorates one or more of the disadvantages or problems described above, or which at least provides a useful alternative.

Other preferred objects of the present invention will become apparent from the following description.

### SUMMARY OF INVENTION

In one form, although it need not be the only or indeed the broadest form, there is provided a hand-held dynamometer comprising:
a body having a first end and a second end opposite the first end;
a first connector at the first end configured to releasably attach a first accessory;
a second connector at the second end configured to releasably attach a second accessory;
at least one force sensor configured to measure compression forces between the first accessory and the body or tension forces between the first accessory and the body and/or compression forces between the second accessory and the body or tension forces between the second accessory and the body; and
a processor configured to receive sensor data from the at least one force sensor and output at least one force measurement to one or more of a display or external device.

The first connector and second connector may be the same such that the first accessory and second accessory can both be attached at either the first or end or the second end of the body. The first and second connectors are preferably aligned along the same axis. That axis may be a longitudinal axis of the body. The body may be substantially cylindrical with the first end and second end being at opposite ends of the cylinder.

The first and second connectors may comprise a releasable locking mechanism. The first and second connectors may comprise a bayonet mount. In use, the bayonet mount may comprise longitudinal movement along an insertion axis followed by rotation around the insertion axis to attach the accessory to the body. Removal of the accessory from the body may be in reverse. The insertion axis may correspond to a longitudinal axis of the body. The insertion axis may correspond to an axial axis of the accessory. The locking mechanism may comprise a clip on the body that engages with a catch on the accessory once the accessory has been rotated sufficiently with respect to the body. The clip may be depressed to release the catch during removal.

The first and second accessories may comprise a plurality of prongs or keyways. The first and second connectors may comprise a plurality of corresponding keyways or prongs, respectively. The keyways may be configured to receive and retain the prongs therein. The prongs may comprise enlarged portions. The prongs may comprise a flanged end. The prongs may be pins with the flange extending radially therefrom. The keyways may comprise a curved channel with an enlarged aperture at one end. There may be two keyways spaced equidistantly from a central longitudinal axis of the body.

The accessories may comprise an electronically readable identifier. The processor may be further configured to determine a type of accessory attached to the body. The electronically readable identifier may be read wirelessly by a reader located in the body. The electronically readable identifier may comprise near field communication (NFC). The electronically readable identifier may comprise radio frequency identification (RFID). The electronically readable identifier may be passive. The electronically readable identifier may be active. An active electronically readable identifier may be powered by a power source in the accessory. Alternatively, it may be powered from the body when connected thereto. The electronically readable element may comprise a light and/or colour and/or hall effect sensor.

The processor may be further configured to predict the exercise being performed based, at least in part, upon its determination of the type of accessory attached to the body. The processor may be further configured to predict the exercise being performed based, at least in part, upon the sensor data received from the force sensors. The processor may be further configured to display at least one of a description and status of the accessory upon its attachment to the body.

The hand-held dynamometer may further comprise an inertial measurement unit (IMU) in communication with the processor. The processor may be further configured to predict the exercise being performed based, at least in part, upon movement data communicated to the processor from the IMU. The IMU may be a 9 axis IMU configured with the processor to determine an angle of movement of the body during use. The IMU may comprise a three-axis gyroscope, a three-axis accelerometer, and a 3-axis compass.

The hand-held dynamometer may further comprise a strap configured to affix the body to a limb of a user during use. The strap may be adjustable. The strap may comprise hook and loop fastening. The strap may be removable.

The external device may comprise a tablet, a smart phone, a computer, an external display, a TV, or the like. The processor may be in electronic communication with a wireless interface, such as Bluetooth, to communicate with the external device.

In another form, there is provided an accessory, preferably for use with the hereinbefore described hand-held dynamometer, the accessory comprising:
an accessory connector configured to connect with, and be retained by, the first connector or second connector of the body of the dynamometer;
an electronically readable identifier configured to communicate with a reader located in the body of the dynamometer when connected thereto; and
one of a pad, hook, loop.

In another form, there is provided a grip strength accessory, preferably for use with the hereinbefore described hand-held dynamometer, the accessory comprising:
a base having a first accessory connector configured to connect with the first connector of the body of the dynamometer;
a frame extending from the base, past the second end of the body of the dynamometer when in use, to define an opening adjacent to its second connector; and
a handle located within the opening and movable relative to the frame, the handle having a second accessory connector configured to connect with the second connector of the body of the dynamometer such that a user can apply tensile force to the dynamometer by pulling the handle towards the frame.

In another form, there is provided a method of using a hand-held dynamometer, the method comprising the steps of:
attaching a first accessory to a first connector located at a first side of a body;
attaching a second accessory to a second connector located at a second side of the body;
performing a first test or exercise with the dynamometer;
taking measurements relating to the first test or exercise with the dynamometer;
replacing the first accessory with a third accessory;
performing a second test or exercise with the dynamometer; and
taking measurements relating to the second test or exercise with the dynamometer;
wherein the measurements from the first test and second test can comprise either of compression force and/or tension force.

The method may further comprise determining a type of accessory upon attachment to the body by reading an electronically readable element of the accessory. The method may further comprise automatically determining a test or exercise being performed based, at least in part, upon the determination of the type of accessory attached to the body of the dynamometer. The measurements from the first test and second test may also comprise angle of movement of the dynamometer.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a front perspective view of a dynamometer with accessories attached;

FIG. 2 illustrates a rear perspective view of the dynamometer of FIG. 1;

FIG. 3 illustrates a front perspective view of the dynamometer of FIG. 1 with accessories detached;

FIG. 4 illustrates a rear perspective view of the dynamometer of FIG. 1 with accessories detached;

FIG. 5 illustrates a front end perspective view of the dynamometer body of FIGS. 1 to 4;

FIG. 6 illustrates an exploded perspective view of the curved pad accessory illustrated in FIGS. 1 to 4;

FIG. 7 illustrates an exploded perspective view of the flat pad accessory illustrated in FIGS. 1 to 4;

FIG. 8 illustrates a perspective view of a dynamometer with different accessories attached;

FIG. 9 illustrates an exploded perspective view of the dynamometer of FIG. 8;

FIG. 10 illustrates a dynamometer body with a strap accessory;

FIG. 11 illustrates a dynamometer with a grip accessory; and

FIG. 12 illustrates the dynamometer with grip accessory of FIG. 11 in a different configuration.

### DETAILED DESCRIPTION OF THE DRAWINGS

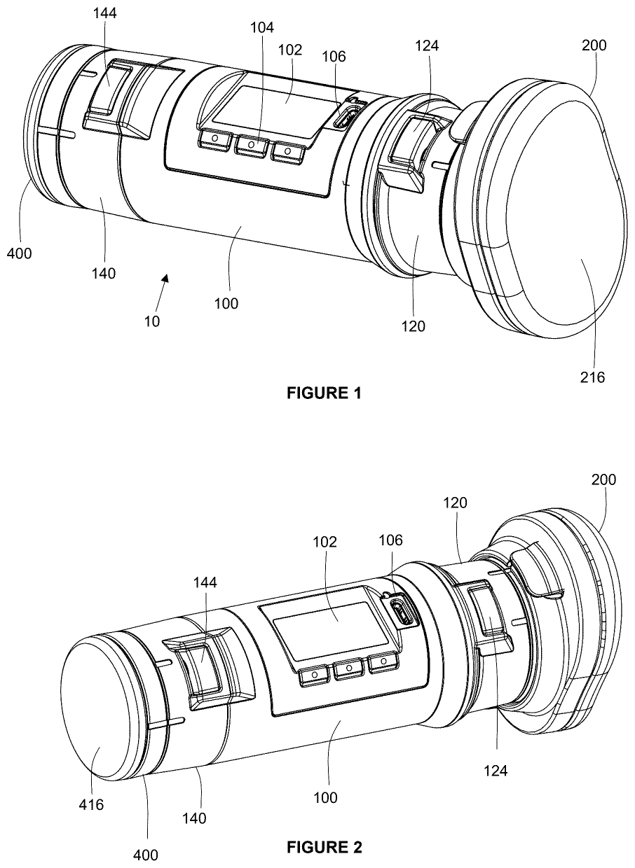

FIGS. 1 and 2 illustrate a dynamometer 10 having a body 100 having a first accessory 200 at a first end 120 and a second accessory 400 at a second end 140. The body 100 is generally cylindrical and has a user interface in the form of a display 102 and buttons 104 located on one side thereof. A charging and/or data port 106 is also provided adjacent the display 102 and buttons 104.

Located inside the body 100 is circuitry with a processor to which the display 102, buttons 104, and charging/data port 106 are electrically connected. Also located inside the body 100 is a power storage device such as a battery configured to provide power to everything connected to the circuitry including, for example, the processor.

The circuitry also comprises one or more force sensors. In preferred forms, there is provided an S-Type load cell capable of measuring both compressive and tensile forces. In preferred forms, the circuitry includes an Inertial Measurement Unit (IMU) connected to the processor. The IMU is preferably a 9-axis IMU having a three-axis gyroscope, a three-axis accelerometer, and a 3-axis compass. The processor and display 102 are preferably configured to be able to display live force measurement data from the load cell during use. The display 102 may also be used to display other relevant data or options for the user which may be selected or changed using the buttons 104.

The circuitry preferably also includes a wireless interface for wireless communication. In preferred forms the wireless interface comprises Bluetooth, which can be used to transmit measurement data, or other relevant information, to an external device such as a tablet, smart phone, computer, external display, TV, etc. In preferred forms the external device is a tablet, smart phone, or computer capable of further processing and two-way communication with the dynamometer 10. The external device may process measurement data received from the dynamometer to automatically detect and display movement and measurements in real time on a screen of the external device. Data from the dynamometer may also be saved for future record keeping and analysis. Such saved data may be able to be stored together with a detected test or exercise type to allow comparison.

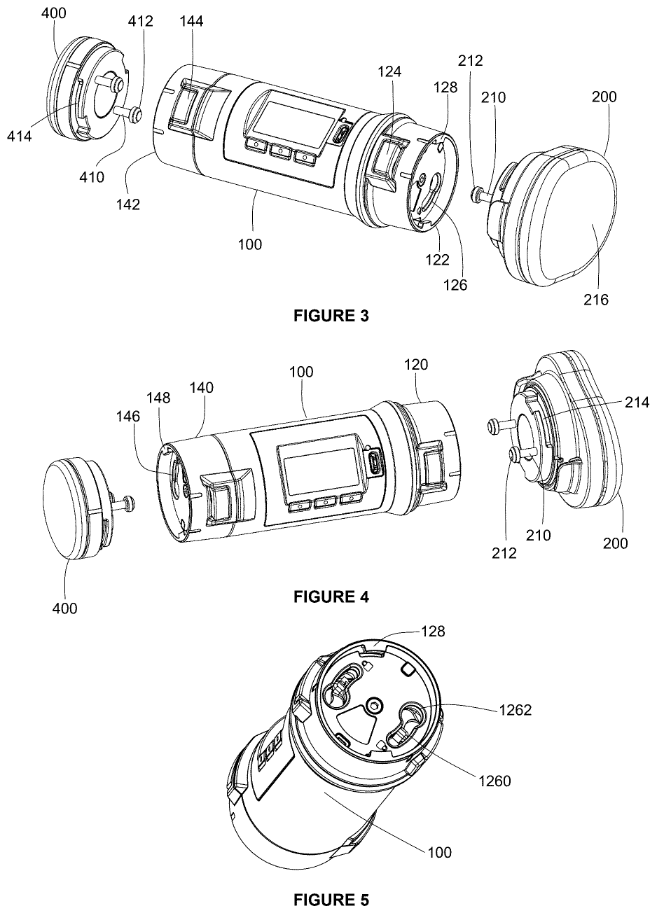

FIGS. 3 and 4 illustrate the dynamometer 10 with the accessories 200, 400 separated from the body 100. As can be seen more clearly in these figures, the first end 120 of the body 100 has a first connector 122 and the second end 140 of the body 100 has a second connector 142. With the connectors 122, 142 the accessories 200, 400 and be attached and released from the body 100 as desired. The accessories 200, 400 are interchangeable and can also be replaced with different accessories as needed (e.g., of different sizes, shapes, and/or function).

The interface between the connectors 122, 142 and accessories 200, 400 is effectively a bayonet mount. In use, prongs 210, 410 of the accessories 200, 400 are received by, and can travel within, keyways 126, 146 of the connectors 122, 142. The connectors 122, 142 also comprise inwardly extending tabs 128, 148 that correspond to grooves 214, 414 in the corresponding connection portion of the accessories 200, 400. The keyways 126, 146 and grooves 214, 414 are of the same arcuate length but, due to being different distances from the axis of rotation of the body 100 and/or accessory 200, 400 during attachment, are of different actual lengths. It should be appreciated that although the prongs 210, 410 and grooves 214, 414 are located on the accessories 200, 400 and the keyways 126, 146 and tabs 128, 148 are located on the body 100 in the illustrated embodiment, that they could be switched such that, for example, the prongs 210, 410 extend from the body 100 into keyways 126, 146 of the accessories 200, 400.

The connectors 122, 142 have a releasable locking mechanism, illustrated in the form of a clip 124, 144, that can be actuated to retain and release the accessories 200, 400 to and from the body 100 as desired. In preferred forms the clip 124, 144 comprises a spring-loaded button. The releasable locking mechanism is preferably biased such that upon attachment of an accessory 200, 400 it is moved to the locked position automatically. The releasably locking mechanism may comprise biased catches which moves by depressing the clip 124, 144 to release the prongs 210, 410 of an accessory 200, 400 during removal.

As can be seen more clearly in FIG. 5, which illustrates the first end 120 of the body 100 in further detail, the keyways 126, 146 comprise a curved channel 1260 with an enlarged aperture 1262. This allows enlarged portions of the prongs 210, 410, in the form of flanged ends 212, 412, to be inserted longitudinally into the keyways 126, 146. Once the flanged ends 212, 412 of the prongs 210, 410 are located in the enlarged apertures 1262 of the keyways 126, 146 the accessory 200, 400 can be rotated around the longitudinal axis such that the flanged ends 212, 412 travel along the channels 1260 of the keyways 126, 146 whereby they can be caught and retained by the locking mechanism 124, 144.

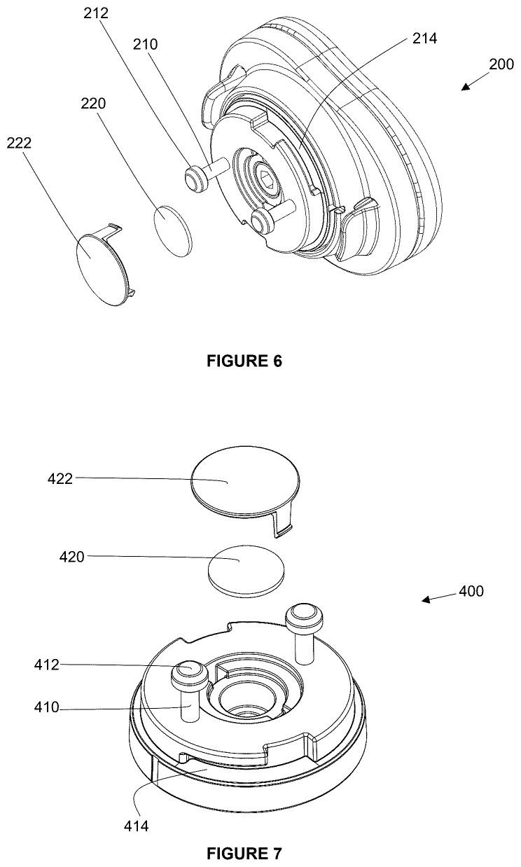

FIGS. 6 and 7 illustrate accessories 200 and 400, respectively, in further detail. Most notably each accessory has an electronically readable identifier 220, 420 located therein. In preferred forms the electronically readable identifier 220, 420 comprises NFC circuitry capable of communicating with the processor in the body 100 of the dynamometer 10 wirelessly when the accessory 200, 400 is attached to the body 100 as shown in FIGS. 1 and 2. A cap or cover 222, 422 is provided to conceal and protect the electronically readable identifier 220, 420 when it is located inside the accessory 200, 400.

As seen more clearly in FIG. 1, the accessory 200 comprises a curved pad 216 used to measure compression forces using sensors located inside the body 100 of the dynamometer. The curved pad 216 of accessory 200 is designed to suit the contour of a curved surface, such as the shin, forearm or heel of a user or subject. The curved pad 216 preferably comprises rubber with a resistance selected to maintain integrity while providing a comfortable pad for human contact. In another accessory the curved pad 216 may be flat (not shown) for contact against a larger surface of the user or subject. Accessory 400 is a palm pad designed and contoured to fit within the middle of a user's palm. This allows a user to provide resistance during a compression test.

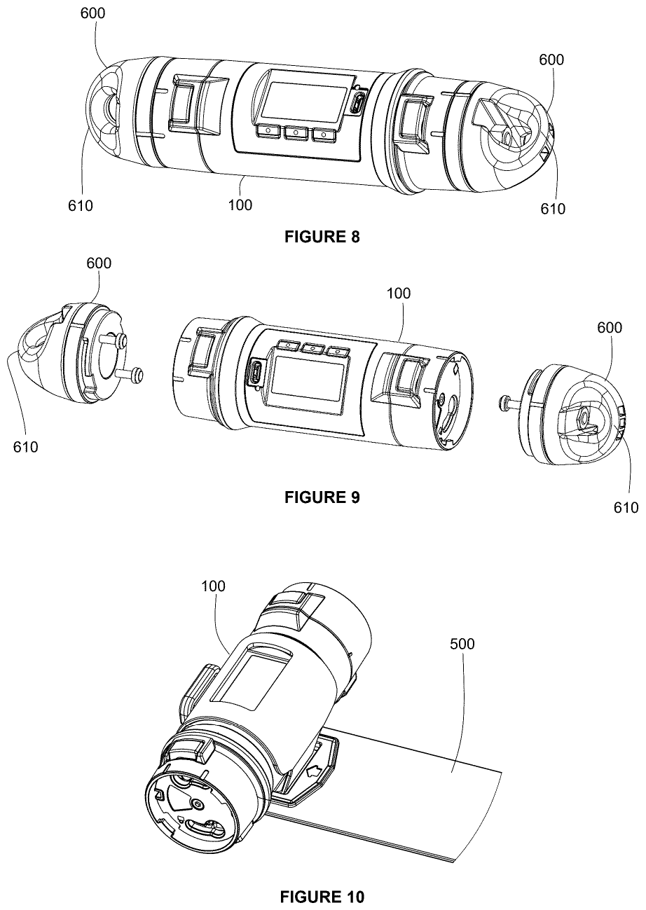

FIGS. 8 and 9 illustrate the body 100 with a tensile accessory 600 on both ends 120, 140 designed for measuring tensile forces. The tensile accessory 600 comprises a loop 610 designed to fit and allow attachment of either a carabiner or strap attachment to perform a tension test. It should be appreciated that a hook, particularly a gated hook (such as a carabiner) could be utilised instead of the loop 610 instead. The tensile accessory 600 may be used, for example, by coupling one end to a fixed resistance such as a physiotherapy bed, and coupling the other end to the user to measure their pulling strength.

FIG. 10 illustrates a strap 500 which can be attached to a limb of a user or patient by encircling their limb with the strap 500. The strap 500 retains the body 100 of the dynamometer and allows a user patient to move freely whilst, for example, the dynamometer measures a range of motion of the limb using its IMU. Data from the IMU can be processed, either on the dynamometer or at an external device, using quaternions to find the maximal angle out of the three planes of movement and determine an angle of movement of the body 100, and hence the angle of movement of the limb of the wearer, during use.

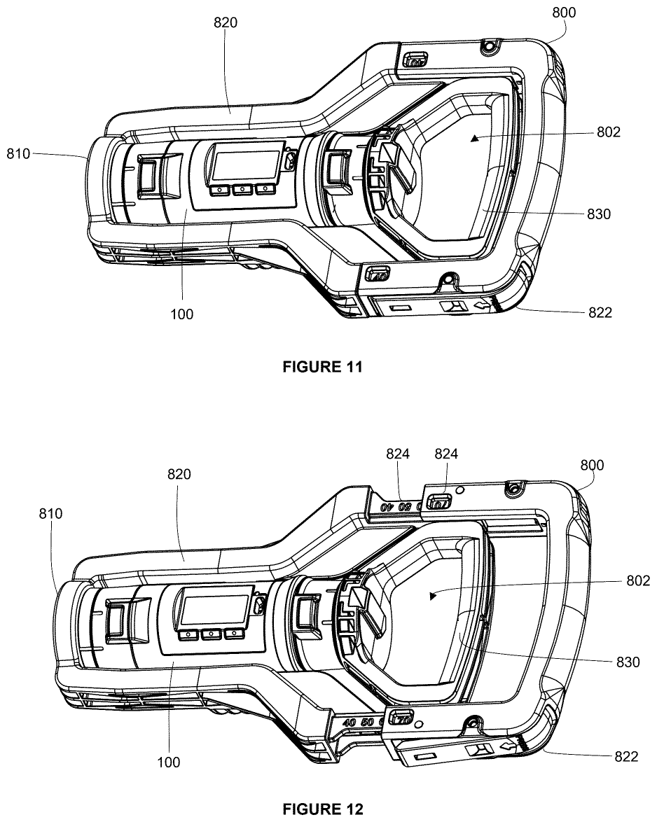

FIGS. 11 and 12 illustrate a grip strength accessory 800 that can retain the body 100 of the dynamometer therein. The grip strength accessory 800 has a base 810 to which a connector 120, 140 of the dynamometer body 100 can be attached (as shown). Extending from the base 810 is a frame 820 that extends past the body 100 and extends radially outward from the longitudinal axis of the body 100 to define an opening 802 within which a handle 830 is located. The handle 830 can be attached to the other connector 122, 142 at the other end 120, 140 of the dynamometer body (as shown).

The frame 820 has an extendable portion 822 which is illustrated in FIG. 11 in an unextended position and in FIG. 12 in an extended position. The extendible portion 822 may be variably extended, preferably with predetermined extension positions. Indicia 824 are provided to indicate how far the extendible portion 822 has been extended. In the illustrated embodiment, the indicia 824 is located on a portion of the frame 820 that is received telescopically within the extendible portion 822. The extendible portion 822 then has a window 826 though which specific indicia 824 on the frame 820 can be viewed. Once the extendible portion 822 has been set at an extension of interest, a user may pull the handle 830 towards the extended end of the frame 822 to measure their grip strength with the dynamometer measuring tensile forces between its connection to the frame 820 at one end and the handle 830 at the other.

In use, the dynamometer 10 can be easily transported to a location for use and configured with a large variety of different accessories to be able to conduct almost all dynamometry and range of motion tests required of a practitioner or user. A single multi-use device, the dynamometer 10, can replace a plurality of traditional devices, greatly simplifying equipment needs for a practitioner or user.

Advantageously, the dynamometer 10 is modular with its connectors and allows the same attachment mechanism for many different types of accessories. The dynamometer 10 can measure both compressive forces and tensile forces which, when coupled with a variety of different accessories, allows for measurement and/or assessment of many different exercises or tests. In addition to allowing for different exercises or tests, different sized and shaped accessories can accommodate a wider range of users and environments. For example, in some situations it may be necessary to use the dynamometer between a physician and a patient. In another, it may be possible or necessary to use the dynamometer between a rigid surface, such as a wall or bench, and the patient. The hand-held nature of the dynamometer allows it to be portable and used in the field at an affordable cost.

The addition of an IMU able to measure range of motion of a limb of a user or patient allows the dynamometer to be able to select and measure over 300 different strength and range of motion tests. Test data is automatically detected and saved against each specific test type, movement type, limb, and position. Test data can be transmitted (either from the dynamometer 10 itself or from an external device in communication with the dynamometer 10 in use) for storage and/or further in-depth data analysis. The load cell and IMU can also be used simultaneously to measure dynamic strength and provide detailed insight into force production that is not typically available to traditional dynamometers.

By having electronically readable elements 220, 420 located within accessories the dynamometer is able to know which accessories 200, 400 are attached and use this information to predict the type of exercise or test about to be performed. Furthermore, the display 102 can show details about the accessory that is attached and can limit types of measurements that may be recorded (e.g. not measuring compression when the tensile accessories 600 are attached). Prediction of the type of exercise or test also saves the user significant time in configuring the dynamometer and recording the test or exercise data appropriately in an automatic and streamlined manner. It also reduces manual error or omission as the dynamometer or external device is able to save tagged measurement data against specific accessories and/or predicted exercises. Once an accessory combination is known, the dynamometer and/or external device can provide suggested tests or exercises to the user. More suitably in the case of an external device, the user can be provided with test or exercise options and instructions for performing them.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step etc.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

As used herein, an element or operation recited in the singular and preceded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

1. A hand-held dynamometer comprising:
a body having a first end and a second end opposite the first end;
a first connector at the first end configured to releasably attach a first accessory;
a second connector at the second end configured to releasably attach a second accessory;
at least one force sensor configured to measure compression forces between the first accessory and the body and tension forces between the first accessory and the body and compression forces between the second accessory and the body and tension forces between the second accessory and the body; and
a processor configured to receive sensor data from the at least one force sensor and output at least one force measurement to one or more of a display or external device.

2. The hand-held dynamometer of claim 1, wherein the first connector and second connector are the same such that the first accessory and second accessory can both be attached at either the first end or the second end of the body.

3. The hand-held dynamometer of claim 1, wherein the first and second connectors comprise a releasable locking mechanism.

4. The hand-held dynamometer of claim 3, wherein the first and second connectors comprise a bayonet mount.

5. The hand-held dynamometer of claim 1, wherein the first and second accessories comprise a plurality of prongs or keyways and the first and second connectors comprise a plurality of corresponding keyways or prongs, respectively, whereby the keyways are configured to receive and retain the prongs therein.

6. The hand-held dynamometer of claim 5 wherein the prongs comprise a flanged end.

7. The hand-held dynamometer of claim 1, wherein the accessories comprise an electronically readable identifier and the processor is further configured to determine a type of accessory attached to the body.

8. The hand-held dynamometer of claim 7, wherein the electronically readable identifier can be read wirelessly by a reader located in the body.

9. The hand-held dynamometer of claim 7, wherein the electronically readable identifier comprises one or more of a near field communication (NFC) or a radio frequency identification (RFID) antennae.

10. The hand-held dynamometer of claim 7, wherein the processor is further configured to predict the exercise being performed based, at least in part, upon its determination of the type of accessory attached to the body.

11. The hand-held dynamometer of claim 7, wherein the processor is further configured to display at least one of a description and status of the accessory upon its attachment to the body.

12. The hand-held dynamometer of claim 1, further comprising an inertial measurement unit (IMU) in communication with the processor.

13. The hand-held dynamometer of claim 12, wherein the IMU is a 9 axis IMU configured with the processor to determine an angle of movement of the body during use.

14. The hand-held dynamometer of claim 1, further comprising a strap configured to affix the body to a limb of a user during use.

15. An accessory for use with the hand-held dynamometer of claim 1, the accessory comprising:
an accessory connector configured to connect with, and be retained by, the first connector or second connector of the body of the dynamometer;
an electronically readable identifier configured to communicate with a reader located in the body of the dynamometer when connected thereto; and
one of a pad, hook, loop.

16. A grip strength accessory for use with the hand-held dynamometer of claim 1, the accessory comprising:
a base having a first accessory connector configured to connect with the first connector of the body of the dynamometer;
a frame extending from the base, past the second end of the body of the dynamometer when in use, to define an opening adjacent to its second connector; and
a handle located within the opening and movable relative to the frame, the handle having a second accessory connector configured to connect with the second connector of the body of the dynamometer such that a user can apply tensile force to the dynamometer by pulling the handle towards the frame.

17. A method of using a hand-held dynamometer, the method comprising the steps of:
attaching a first accessory to a first connector located at a first side of a body;
attaching a second accessory to a second connector located at a second side of the body;
performing a first test or exercise with the dynamometer;
taking measurements relating to the first test or exercise with the dynamometer;
replacing the first accessory with a third accessory;
performing a second test or exercise with the dynamometer; and
taking measurements relating to the second test or exercise with the dynamometer;
wherein the measurements from the first test and second test can comprise either of compression force and/or tension force.

18. The method of claim 17, further comprising determining a type of accessory upon attachment to the body by reading an electronically readable element of the accessory.

19. The method of claim 18, comprising automatically determining a test or exercise being performed based, at least in part, upon the determination of the type of accessory attached to the body of the dynamometer.

20. The method of claim 17, wherein the measurements from the first test and second test further comprise the angle of movement of the dynamometer.
